(19) United States
(12) Patent Application Publication
(10) Pub. No.: US 2025/0268859 A1
(43) Pub. Date: Aug. 28, 2025

(54) OXAFURAMINE, (1R)-N-ETHYL-1-[(2R)-OXOLAN-2-YL]-2-PHENYLETHANAMINE, HYDROCHLORIDE AND DERIVATIVES THEREOF FOR TREATING ALZHEIMER'S DISEASE (AD), VASCULAR DEMENTIA, DEMENTIA WITH LEWY BODIES (DLB), MIXED DEMENTIA, FRONTOTEMPORAL LOBAR DEGENERATION (FTLD), AND PARKINSON'S DISEASE (PD)

(71) Applicant: NLS Pharmaceutics AG, Zurich (CH)

(72) Inventor: Eric KONOFAL, Senlis (FR)

(73) Assignee: NLS Pharmaceutics AG, Zurich (CH)

(21) Appl. No.: 18/576,973

(22) PCT Filed: Jul. 8, 2022
(86) PCT No.: PCT/EP2022/069200

§ 371 Date: Jan. 5, 2024

(30) Foreign Application Priority Data
Jul. 8, 2021 (EP) 21305945.4

(51) Int. Cl. A61K 31/34; A61P 25/28

(52) U.S. Cl. CPC A61K 31/34; A61P 25/28

(57) ABSTRACT

The invention concerns a compound of formula (I) (I) R₁═H, —CH₃ or acyl group, preferably R₁═—CH₃R₂═H or halogen atom selected in the group consisting of: F, Cl, Br, I, preferably R₂═H or a pharmaceutically acceptable isomer, salt and/or solvate thereof, for use in preventing and/or treating neurodegenerative diseases when central muscarinic neurotransmission is compromised, wherein said neurodegenerative disease is selected in the group consisting of Alzheimer's Disease (AD), vascular dementia, Dementia with Lewy bodies (DLB), mixed dementia, frontotemporal lobar degeneration (FTLD), and Parkinson's disease (PD).

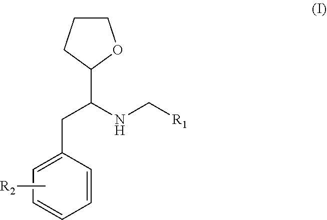

### FIELD OF THE PRESENT INVENTION

Oxafurarmine, (1R)—N-ethyl-1-[(2R)-oxolan-2-yl]-2-phenylethanamine, hydrochloride and derivatives thereof for treating neurodegenerative diseases when central muscarinic neurotransmission is compromised wherein said neurodegenerative disease is selected in the group consisting of Alzheimer's Disease (AD), vascular dementia, Dementia with Lewy bodies (DLB), mixed dementia, frontotemporal lobar degeneration (FTLD), and Parkinson's disease (PD).

### BACKGROUND OF THE PRESENT INVENTION

Dementia is one of the major causes of disability and mortality and a common disease in the elderly. It is characterized by difficulties with memory, language, problem-solving, and a decline in cognitive level, which affects daily routine and social activities. Dementia has different types including Alzheimer's Disease (AD), vascular dementia, Dementia with Lewy bodies (DLB), mixed dementia, frontotemporal lobar degeneration (FTLD), and Parkinson's disease (PD) dementia^{1,2}.

AD is the most common type of dementia. There are about 10 million new cases annually and AD may contribute to 60-70% of the cases.

DLB accounts for 15-25% of dementia in the elderly. In DLB, there is a spectrum of pathology along the interface between PD and AD. Many DLB patients have the neuropathological features of AD, including senile plaques and neurofibrilliary tangles.

Alterations in cholinergic functions have been reported to be associated with neuropsychiatric symptoms in dementia^{3,4}.

Moreover, in AD, PD and DLB, muscarinic receptors M₁ are altered in different areas⁵ when M₁ receptor density has been reported to be moderately reduced in cortex in AD^{6,7}, particularly in the hippocampus, and to be elevated in the striatum in AD⁸. Also, M₁ receptors are of high density in cortex and striatum and are relatively low in the thalamus and cerebellum⁵.

Increased M₁ muscarinic receptor binding in temporal cortex is associated with delusions in DLB patients, when increased M₂ binding was significantly associated with increased M₂ binding³.

An increasing M₂ and M₄ receptor binding has been associated with visual hallucinations in patients with DLB targeting on M₂ receptors antagonists as possible treatment of DLB symptomatology^{3,5,9}.

M₁ receptors are of high density in cortex and striatum and are relatively low in the thalamus and cerebellum, while M₄ receptors are mainly expressed in the striatum⁵. M₄ receptor was found to be the main subtype expressed in rat striatal projection neurones, forming 50% of total muscarinic receptors and co-localising with dopamine D₁ receptors¹⁰, while M₁ receptors were located on D₂ bearing striato-pallidal neurons^{10,11}.

Muscarinic M₅ receptors are selectively enriched in the Substantia Nigra (SN) and Ventral Tegmental Areas of rat brain, suggesting that they may have a role in the modulation of dopaminergic transmission¹² when muscarinic imodulation of mesolimbic dopanilnergic neurons in the ventral tegmental area (VTA) plays an important role in reward, potentially mediated through the M₅ muscarinic acetylcholine receptor¹³.

Because M₅ muscarinic receptors are the only muscarinic receptor subtype associated with VTA and SN dopamine neurons, in associating dopamine reuptake inhibition in VTA and cholinergic enhancing by decreasing M1 receptor binding in temporal cortex and M4 receptor binding in adjacent (BA 32) and cingular cortex, and because also VTA and the rostromedial tegmental nucleus (RMTg) each contribute to opiate reward and each receive inputs from the laterodorsal tegmental and pedunculopontine tegmental nuclei, drug acting on these receptors and fonctions may have potential implications for opiods-induced dependence treatment^{14,15}.

Oxafuramine is a stimulant considered as a potential candidate for treating neurodegenerative diseases when central muscarinic neurotransmission is compromised as Dementia with Lewy bodies (DLB) and/or Alzheimer's Disease (AD).

The pharmacological binding profile of Oxafuramine has never been studied and/or published.

Oxafuramine, (1R)—N-ethyl-1-[(2R)-oxolan-2-yl]-2-phenylethanamine, is a psychostimulant acting on dopamine transporter (DAT) and norepinephrine transporter (NET) as inhibitor at 10⁻⁵ M concentration.

Oxafuramine, (1R)—N-ethyl-1-[(2R)-oxolan-2-yl]-2-phenylethanamine presenting muscarinic M₄/M₁/M₅ receptors antagonist activities, which are approximatively 51%, 30% and 25% at 10⁻⁵ M (Table 1) is a cholinergic modulator and cognitive enhancer. M₄ more than M₁ and M₅ receptors antagonists have been shown to improve cognition requirement and motor control and potentially useful in treatment of behavioral disorders and neurodegenerative disorders.

### SUMMARY OF THE PRESENT INVENTION

An object of the invention is a compound of formula (I)

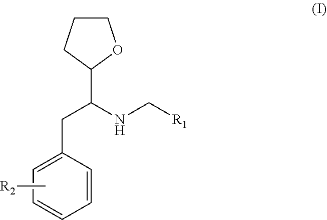

R₁=H, —CH₃ or acyl group
R₂═H or halogen atom selected in the group consisting of F, Cl, Br, I,
   or a pharmaceutically acceptable isomer, salt and/or solvate thereof, for use in preventing and/or treating neurodegenerative diseases when central muscarinic neurotransmission is compromised, wherein said neurodegenerative disease is selected in the group consisting of Alzheimer's Disease (AD), vascular dementia, Dementia with Lewy bodies (DLB), mixed dementia, frontotemporal lobar degeneration (FTLD), and Parkinson's disease (PD).

Another object of the invention is a pharmaceutical composition comprising a compound of formula (1) or a pharmaceutically acceptable isomer, salt and/or solvate thereof as defined in claim 1 and a pharmaceutically acceptable excipient for use preventing and/or treating neurodegenerative diseases when central muscarinic neurotransmission is compromised wherein said neurodegenerative disease is selected in the group consisting of Alzheimer's Disease (AD), vascular dementia, Dementia with Lewy bodies (DLB), mixed dementia, frontotemporal lobar degeneration (FTLD), and Parkinson's disease (PD).

### FIGURES

FIG. 1 Time schedule of the test.

FIG. 2 Effects of donepezil and NLS-12 on the discrimination index (DI; mean±SEM and individual values). Difference vs. Control group: ns=not significant; *p≤0.05; **p≤0.01

Difference vs. Donep 2 group: not significant in all cases except for Control group (not represented)
Difference vs. 0: #p≤0.05; ###p≤0.001; Otherwise: not significant

FIG. 3 Effects of donepezil NLS-12 on the difference of exploration time between the novel object and the familiar object (N-F; mean±SEM and individual values). Difference vs. Control group: ns=not significant; *p≤0.05; **p≤0.01.

Difference vs. Donep 2 group: not significant in all cases except for Control group (not represented)
Difference vs. 0: #p≤0.05; ##p≤0.01; ###p≤0.001; Otherwise: not significant

FIG. 4 Effects of donepezil and NLS-12 on the exploration time during the sample trial (ST; mean±SEM and individual values). Comparisons vs. the Control group. Difference vs. Control group: ns=not significant; **p≤0.01.

FIG. 5 Effects of donepezil and NLS-12 on the exploration time during the sample trial (ST; mean±SEM and individual values). Comparisons vs. the Donepezil 2 group. Difference vs. Donep 2 group: ns=not significant; *p≤0.05; **p≤0.01; ***p≤0.001.

FIG. 6 Effects of donepezil and NLS-12 on the exploration time during the choice trial (CT=N+F; mean±SEM and individual values). Difference vs. Control group: ns=not significant. Difference vs. Donep 2 group: not significant in all cases (not represented).

### DETAILED DESCRIPTION OF THE PRESENT INVENTION

The first subject-matter of the invention relates to a compound of formula (I)

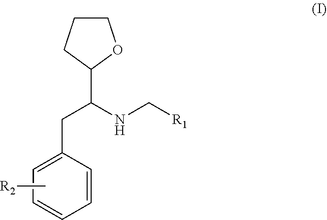

R₁=H, —CH₃ or acyl group, preferably R₁═—CH₃,
R₂═H or halogen atom selected in the group consisting of: F, Cl, Br, I, preferably R₂═H, or a pharmaceutically acceptable isomer, salt and/or solvate thereof, for use in preventing and/or treating neurodegenerative diseases when central muscarinic neurotransmission is compromised, wherein said neurodegenerative disease is selected in the group consisting of Alzheimer's Disease (AD), vascular dementia, Dementia with Lewv bodies (DLB), mixed dementia, frontotemporal lobar degeneration (FTLD), and Parkinson's disease (PD).

R₂ is in meta, ortho or para position.

Formula (I) has a chiral center.

Thus, “isomer” means preferably “enantiomer”.

According to the present invention, and when not specified otherwise, the term “compound of formula (I)” refers to compound of formula (I) in its racemic form or in its enantiomeric forms.

An “optically pure compound of formula (I)” means an enantiomer in an enantiomeric excess of more than 95%, preferably of more than 96%, more preferably of more than 97%, even more preferably of more than 98%, particularly preferably of more than 99%.

When R₁═—CH₃, R₂═H, optically pure R-enantiomer, compound of formula (I) is oxafuramine, (1R)—N-ethyl-1-[(2R)-oxolan-2-yl]-2-phenylethanarnine, its salts, in particular its hydrochloride salt.

Preferably, said neurodegenerative diseases has different types including Alzheimer's Disease (AD), vascular dementia, Dementia with Lewy bodies (DLB), mixed dementia, frontotemporal lobar degeneration (FTLD), and Parkinson's disease (PD).

Compound of formula (I) is preferably used at a therapeutic dose comprised between 0.1 mg/kg/day and 400 mg/kg/day is administrated to a patient in need thereof, more preferably between 2 and 128 mg/kg/day.

The second subject-matter of the invention relates to a method of prevention and/or treatment of neurodegenerative diseases when central muscarinic neurotransmission is compromised), comprising the administration of a compound of formula (I) as defined above or a pharmaceutically acceptable isomer, salt and/or solvate thereof, to a patient in need thereof, wherein said neurodegenerative disease is selected in the group consisting ofAlzheiner's Disease (AD), vascular dementia, Dementia with Lewy bodies (DLB), mixed dementia, frontotemporal lobar degeneration (FTLD), and Parkinson's disease (PD).

The third subject-matter of the invention relates to a pharmaceutical composition comprising a compound of formula (I) or a pharmaceutically acceptable isomer, salt and/or solvate thereof as defined above and a pharmaceutically acceptable excipient for use in preventing and/or treating neurodegenerative diseases when central muscarinic neurotransmission is compromised, wherein said neurodegenerative disease is selected in the group consisting of Alzheimer's Disease (AD), vascular dementia, Dementia with Lewy bodies (DLB), mixed dementia, frontotemporal lobar degeneration (FTLD), and Parkinson's disease (PD).

Preferably, the pharmaceutical composition for use according to the invention comprises between 1 mg to 80 mg, preferably 2 mg to 40 mg of compound of formula (I).

Preferably, the pharmaceutical composition for use according to the invention is suitable for oral administration, for example in the form of a tablet, a capsule, a syrup, a solution, a powder or parenteral administration, for example in the form of a solution, such as an injectable solution and for transdermal system (TDS).

### Examples

Oxafuramine is tested at 10⁻⁵ M, calculated as a % inhibition of control specific binding of a radioactively labeled ligand specific for each target.

This binding profile panel was broadly defined with roughly an equal number of selective, central and peripheral therapeutically relevant targets, including native animal tissues, radioligands and specific enzymes involved in cell cycle regulation in accordance with Eurofins Standard Operating Procedure (www.eurofins.fr).

For radioligand binding experiments, the half maximal inhibitory concentration (IC₅₀) and the half maximal effective concentration (EC₅₀) values were determined (via computer software) by nonlinear regression analysis of the competition curves using Hill equation curve fitting. The inhibition constants (Kᵢ) were calculated using the Cheng-Prusoff equation (Kᵢ=IC₅₀/(1+(L/K_{D})), where L is the concentration of radioligand in the assay, and K_{D} is the affinity of the radioligand for the receptor.

The results are expressed as a % control specific binding ([measured specific binding/control specific binding]×100) and as a % inhibition of control specific binding (100−[(measured specific binding/control specific binding)×100]) obtained in the presence of the test compounds.

Results showing an inhibition (or stimulation) lower than 25% are not considered significant and mostly attributable to variability of the signal around the control level.

Low to moderate negative values have no real meaning and are attributable to variability of the signal around the control level.

An inhibition or stimulation of more than 50% is considered a significant effect of the test compounds and between 25% and 50% indicated of weak to moderate effects that should be confirmed by further testing as they are within a range where more inter-experimental variability can occur.

Fifty percent is a common cut-off for further investigation (i.e. determination of IC₅₀ or EC₅₀ values from concentration-response curves).

Principal significant or pertinent findings of these binding assays are respectively presented for Oxafuramine in Table 1.

TABLE 1
Binding activity sites for oxafuramine

| | % Inhibition at 10⁻⁵M |
|---|---|
| Assay | Oxafuramine |
| DAT (h) (antagonist radioligand) | 94.1 |
| NET (h) (antagonist radioligand) | 53.3 |
| M₄ (h) (antagonist radioligand) | 50.8 |
| M₁ (h) (antagonist radioligand) | 29.7 |
| M₅ (h) (antagonist radioligand) | 25.4 |
| GABA-A₁ (antagonist radioligand) | 19.2 |
| Relaxin-3 (RXFP3) (h) (agonist radioligand) | 16.5 |

The principal result of these binding assays confirmed that Oxafuramine exhibited appreciable potencies for dopamine transporter (DAT) and norepinephrine transporter (NET) at 10⁻⁵ M concentration. Also, Oxafuramine presented muscarinic M₄/M₁/M₅ receptors antagonist activities, which are approximatively 51%, 30% and 25% at 10⁻⁵ M (Table 1). M₄ more than M₁ and M₅ receptors antagonists have been shown to improve cognition requirement and motor control and potentially useful in treatment of behavioral disorders and dementia diseases¹⁶.

The relaxin family peptide receptor 3 (relaxin-3/RXFP3), a G protein-coupled receptor (GPCR) widely expressed in the cortex and involved in stress responses and memory and emotional processing and AD¹⁷ is found to weakly target by Oxafuramine (Study US073-0006869-Q Eurofins/leadHunter Aug. 1, 2019; unpublished data) (Table 1).

### Material and Methods

In these assays' compounds were tested in agonist and antagonist mode with the GPCR Biosensor Assays and match to this design:

### Cell Handling

1. cAMP Hunter cell lines were expanded from freezer stocks according to standard procedures.
2. Cells were seeded in a total volume of 20 μL into white walled, 384-well microplates and incubated at 37° C. for the appropriate time prior to testing.
3. cAMP modulation was determined using the DiscoverX HitHunter cAMP XS+ assay.

### Gs Agonist Format

1. For agonist determination, cells were incubated with sample to induce response.
2. Media was aspirated from cells and replaced with 15 μL 2:1 HBSS/10 mM Hepes: cAMP XS+ Ab reagent.
3. Intermediate dilution of sample stocks was performed to generate 4× sample in assay buffer.
4. 5 μL of 4× sample was added to cells and incubated at 37° C. or room temperature for 30 or 60 minutes. Vehicle concentration was 1%.

### Gi Agonist Format

1. For agonist determination, cells were incubated with sample in the presence of EC80 forskolin to induce response.
2. Media was aspirated from cells and replaced with 15 μL 2:1 HBSS/10 mM Hepes: cAMP XS+ Ab reagent.
3. Intermediate dilution of sample stocks was performed to generate 4× sample in assay buffer containing 4× EC80 forskolin.
4. 5 μL of 4× sample was added to cells and incubated at 37° C. or room temperature for 30 or 60 minutes. Final assay vehicle concentration was 1%.

### Allosteric Modulation Format

1. For allosteric determination, cells were pre-incubated with sample followed by agonist induction at the EC20 concentration.
2. Media was aspirated from cells and replaced with 10 μL 1:1 HBSS/10 mM Hepes: cAMP XS+ Ab reagent.
3. Intermediate dilution of sample stocks was performed to generate 4× sample in assay buffer.
4. 5 μL of 4× compound was added to the cells and incubated at room temperature or 37° C. for 30 minutes.
5. 5 μL of 4× EC20 agonist was added to the cells and incubated at room temperature or 37° C. for 30 or 60 minutes. For Gi-coupled GPCRs, EC80 forskolin was included.

### Inverse Agonist Format (Gi Only)

1. For inverse agonist determination, cells were pre-incubated with sample in the presence of EC20 forskolin.
2. Media was aspirated from cells and replaced with 15 μL 2:1 HBSS/10 mM Hepes: cAMP XS+Ab reagent.
3. Intermediate dilution of sample stocks was performed to generate 4× sample in assay buffer containing 4× EC20 forskolin.
4. 5 μL of 4× sample was added to cells and incubated at 37° C. or room temperature for 30 or 60 minutes. Final assay vehicle concentration was 1%.

### Antagonist Format

1. For antagonist determination, cells were pre-incubated with sample followed by agonist challenge at the EC80 concentration.
2. Media was aspirated from cells and replaced with 10 μL 1:1 HBSS/Hepes: cAMP XS+Ab reagent.
3. 5 μL of 4× compound was added to the cells and incubated at 37° C. or room temperature for 30 minutes.
4. 5 μL of 4× EC80 agonist was added to cells and incubated at 37° C. or room temperature for 30 or 60 minutes. For Gi coupled GPCRs, EC80 forksolin was included.

### Signal Detection

1. After appropriate compound incubation, assay signal was generated through incubation with 20 μL cAMP XS+ED/CL lysis cocktail for one hour followed by incubation with 20 μL cAMP XS+ EA reagent for three hours at room temperature.
2. Microplates were read following signal generation with a PerkinElmer Envision™ instrument for chemiluminescent signal detection.

### Data Analysis

1. Compound activity was analyzed using CBIS data analysis suite (ChemInnovation, CA).
2. For Gs agonist mode assays, percentage activity is calculated using the following formula:

$$\% Activity = 100 \% \times ( mean RLU of test sample - mean RLU of vehicle control ) / ( mean RLU of MAX control - mean RLU of vehicle control ) .$$

3. For Gs positive allosteric mode assays, percentage modulation is calculated using the following formula: http://www.eurofinsdiscoveryservices.com Confidential 6/25/2021

$$5 \% Modulation = 100 \% \times ( mean RLU of test sample - mean RLU of EC 20 control ) / ( mean RLU of MAX control - mean RLU of EC 20 control ) .$$

4. For Gs antagonist or negative allosteric mode assays, percentage inhibition is calculated using the following formula: % Inhibition=100%×(1−(mean RLU of test sample−mean RLU of vehicle control)/(mean RLU of EC80 control−mean RLU of vehicle control)).
5. For Gi agonist mode assays, percentage activity is calculated using the following formula:

$$\% Activity = 100 \% \times ( 1 - ( mean RLU of test sample - mean RLU of MAX control ) / ( mean RLU of vehicle control - mean RLU of MAX control ) ) .$$

6. For Gi positive allosteric mode assays, percentage modulation is calculated using the following formula: % Modulation=100%×(1−(mean RLU of test sample−mean RLU of MAX control) /(mean RLU of EC20 control−mean RLU of MAX control)).
7. For Gi inverse agonist mode assays, percentage activity is calculated using the following formula: % Inverse Agonist Activity=100%×((mean RLU of test sample−mean RLU of EC20 forskolin)/(mean RLU of forskolin positive control−mean RLU of EC20 control)).
8. For Gi antagonist or negative allosteric mode assays, percentage inhibition is calculated using the following formula: % Inhibition=100%×(mean RLU of test sample−mean RLU of EC80 control)/(mean RLU of forskolin positive control−mean RLU of EC80 control).

For agonist and antagonist assays, data was normalized to the maximal and minimal response observed in the presence of control ligand and vehicle.

For Gi cAMP assays, the following forskolin concentration was used:
RXFP3 cAMP: 20 μM Forskolin
RXFP4 cAMP: 20 μM Forskolin

The following EC80 concentrations were used:
RXFP3 cAMP: 0.0003 μM Relaxin-3
RXFP4 cAMP: 0.01 μM Relaxin-3

### Effects of Oxafuramine (NLS-12) and Donepezil on Memory in the Novel Object Recognition (NOR) Test in Mice

### Materials and Methods

### General Points

In this study, Oxafuramine is also called NLS-12.

Manipulations of animals were conducted carefully in order to reduce stress at the minimum. All the experiments were performed in compliance with the guidelines of the French Ministry of Agriculture for experiments with laboratory animals (law 2013-118).

Experiments were conducted in standard conditions (T°=22.0±1.5° C.), with artificial light in quiet conditions (no noise except those generated by ventilation and by the apparatus used for experiments).

Experiments were conducted blindly.

The animals have not been subjected to other experiments before the study.

### Animals

| | |
|---|---|
| Specie, strain, sex | Mice, C57BL/6J, male |
| Age | 6-7 weeks old at the time of testing |
| Number of animals | N = 128 |
| Origin (breeder) | Janvier Labs, France |
| Housing | Group housing (4 mice/cage): 1290D |
| | Eurostandard Type III cages (Tecniplast, Italy) |
| | in transparent polycarbonate (42.5 cm deep; |
| | 26.6 cm large; 15.5 cm high, area = 820 cm²). |
| | Cages are covered with a stainless steel |
| | grid in which food and a bottle are placed. |
| | A stainless steel removable divider |
| | separates food and water. |
| Litter | Aspen Small (SDS Dietex, France) |
| Enrichment | Wood brick, cell huts |
| Temperature | 22.0 ± 1.5° C. |
| Hygrometry | 50 ± 30% (not guaranteed; measured not controlled) |
| Air renewal | Fresh air, 12-25 vol/h |
| Lighting | 20-30 Lux |
| Day/night cycle | 12 h/12 h cycle; light on 8:00-20:00/off: 20:00-8:00 |
| Food | Rat-mouse A04 (Safe, France) available ad libitum |
| Drink | Tap water, available ad libitum |

### Drugs

| | |
|---|---|
| NLS-12 | Oxafuramine |
| Vehicle | 1% CMC + 0.5% Tween 80 in 0.9% NaCl |
| Administration route | Intraperitoneal (i.p.) |
| Doses studied | 1, 4, 8 mg/kg |
| Molecular weight base/ | NT/NT |
| salt | |
| Correction factor | 1 |
| Number of applications | 1 |
| Application volume | 10 ml/kg of body weight. |
| Preparation | Stock solution aliquoted, stocked |
| | at −80° C. for 6 weeks. |
| Storage conditions during | Ambient temperature (22-23° C.) |
| test | |

| | |
|---|---|
| Donepezil | Donepezil hydrochloride; |
| | C₂₄H₃₀ClNO₃; CAS: 120011-70-3 |
| Vehicle | 0.9% NaCl |
| Administration route | Intraperitoneal (i.p.) |
| Doses studied | 2 mg/kg |
| Molecular weight base/ | 379.492/415.95 |
| salt | |
| Correction factor | 1.10 |
| Number of applications | 1 |
| Application volume | 10 ml/kg of body weight. |
| Preparation | Stock solution aliquoted, |
| | stocked at −80° C. for 6 weeks. |
| Storage conditions during | Ambient temperature (22-23° C.) |
| test | |

### Novel Object Recognition (NOR) Test

### Materials

The test was carried out in circular boxes (30 cm diameter, 40 cm high). The objects to be discriminated (L≈1≈h≈3-4 cm) differed in both color and shape and were referred as yellow duck and blue lego. They were fixed with a magnet to the floor of the boxes at 5 cm of the wall, 20 cm distant. Apparently, they have no natural significance for mice and they have never been associated with a reinforcement. In order to rule out the possibility of scent traces left on the objects and therefore the dependency of the recognition capacity of mice on the olfactory cue, the objects and the ground of the box were washed with an odorless disinfectant (Sanicid® diluted in water) and dried between each trial. A camcorder was fixed to the ceiling above the box to record the animals' activity. The experiment was analyzed blindly at a later time.

### Procedure

On the week before the test, animals were handled by the experimenter in charge of the experiment in order to be not stressed at the time of testing. For this purpose, the experimenter placed a small amount of litter, and then the mouse on its hand. Handling took about 1-2 min and was made every day for 4 or 5 consecutive days, and until the mouse did not show any fear to manipulations.

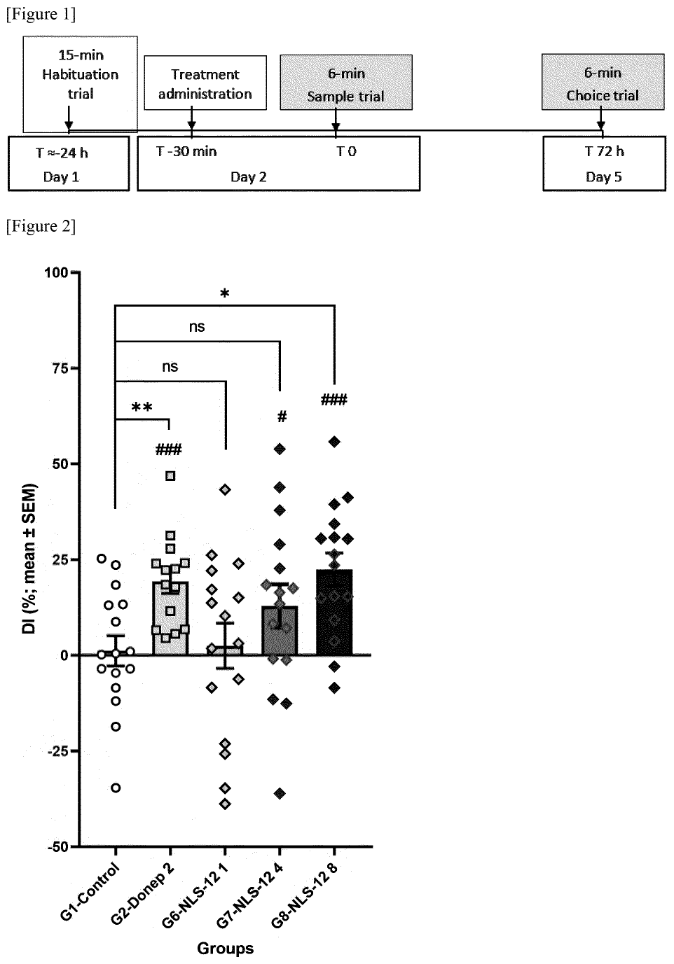

The NOR test was completed over five days (see FIG. 1):
Day 1: Habituation trial. Animals were individually placed for a 15-min free exploration session in empty open boxes.
Day 2. Treatment administration and sample trial. The mice were administered with treatments to which they have been assigned. They were individually placed 30 min after in the apparatus for a 6-min session with two identical objects (Duck, 50% of animals or Lego, 50% of animals).
Day 3. Choice trial. The mice were individually placed for a 6-min session in the apparatus with two objects (Duck and Lego), one of the objects presented in sample trials (termed as familiar objects) and a novel object (Lego, 50% of animals or Duck, 50% of animals).

The sample and choice trials were recorded with the camera located above the apparatus. The time spent by mice in exploring the objects was measured during the sample trial and the choice trial. Exploration of an object was defined as follows: directing the nose to the object at a distance≤2 cm and/or touching it with the nose or forelimbs; turning around or biting the object, or sitting on the object were not considered as exploratory behavior.

### Read-Outs

### Data Recorded:

L=exploration time of the left object in the sample trial.
R=exploration time of the right object in the sample trial.
N=exploration time of the new object in the choice trial.
F=exploration time of the familiar object in the choice trial.

### Object Recognition Task Indices Include the Following Parameters:

Exploration indices:
   ST=L+R=exploration time (left object+right object) in the sample trial.
   CT=N+F=exploration time (new object+familiar object) in the choice trial.
Two memory indices:
   N-F=difference of exploration time between the new object and the familiar object in the choice trial.

$$DI = discrimination index = 100 \times ( N - F ) / ( N + F ) .$$

### Groups

Animals (N=128) were divided into 8 groups (N=16/group) which received 30 min before the sample trial an injection of:
G1-Control group: Vehicle
G2-Donep 2 group: Donepezil (2 mg/kg)
G6-NLS-12 1 group: NLS-12 (1 mg/kg)
G7-NLS-12 4 group: NLS-12 (4 mg/kg)
G8-NLS-12 8 group: NLS-12 (8 mg/kg)

### Data Analysis

Statistical analyses were performed using the GraphPad Prism 9 software.

Data are expressed as mean and standard error of mean (SEM).

A difference is considered statistically significant at p 0.05.

Statistical analyses:
Read-outs: DI, N-F, ST, CT, N, F
   Unpaired Student's t test: Donep 2 group vs. Control group.
   One-way ANOVA, Dunnett test:
      NLS-12 groups vs. Control group.
      NLS-12 groups vs. Donep 2 group.
Read-outs: DI and N-F, for all groups, paired Student's t test, difference vs. 0.
Body weight: one-way ANOVA.

Exclusion criteria: animals which displayed a poor exploratory behavior, i.e. which spent less than 5 sec in exploring the two objects in the sample trial and/or in the choice trial were discarded from the analysis for DI and N-F. All animals were included in the analysis for ST and CT

### Results

Only the most meaningful results, i.e. the effects of treatments on the memory indices (discrimination index and the difference of exploration time between the novel object and the familiar object) and on the exploration indices (exploration time during the sample and choice trials) are described below.

The body weight was not significantly different between groups (ANOVA: F(7;120)=0.9303; p=0.486; see Table).

### Control Group, Effect of Donepezil

The results are presented in Table 2.

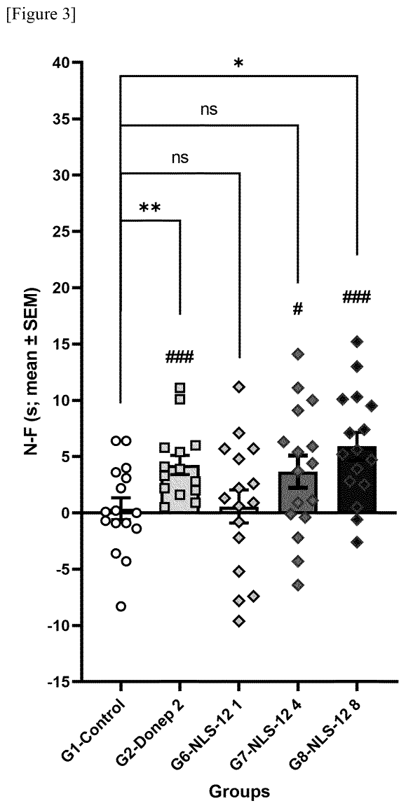

The discrimination index (DI, FIG. 2); and the difference of exploration time between the novel object and the familiar object (N-F; FIG. 3) were not significantly higher than zero for the Control group.

The discrimination index (DI, FIG. 2) and the difference of exploration time between the novel object and the familiar object (N-F; FIG. 3) were significantly higher than zero. Both the DI and the ‘N-F’ were significantly higher in the Donep 2 group than in the Control group.
Summary. The Control group did not recognize the familiar object. Donepezil (2 mg/kg) improved the recognition of the familiar object, i.e. improved memory. Therefore, experimental conditions were suitable to detect an improvement of memory.

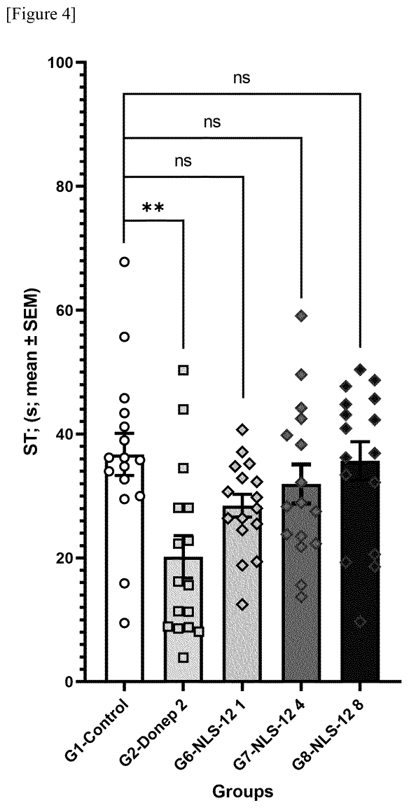

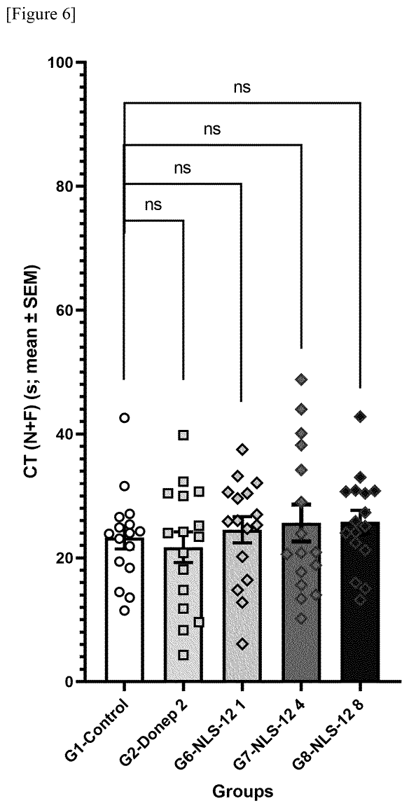

Donepezil (2 mg/kg) decreased the exploration time during the sample trial (ST; 30 min post treatment; FIG. 4) and did not significantly modify the exploration time during the choice trial (CT; 72 h post treatment; FIG. 6).
Summary, Donepezil (2 mg/kg) decreased the exploration time 30 min post-treatment, but not 3 days post treatment.

TABLE 2
Control and Donep 2 groups. Indices of memory: discrimination index (DI) and difference
of exploration time between the novel object and the familiar object (N − F). Indices
of exploration: exploration time during the sample trial (ST) and during the choice trial
(CT). Results are expressed as mean and SEM. Statistical analyses: “p vs. random”, difference
vs. 0 (paired Student's t test); “p vs. G1-Control”, unpaired Student's t test.

| | | DI | N − F | | CT |
|---|---|---|---|---|---|
| | | Discrimination | Exploration time | ST | Exploration time in |
| | | Index = 100 × | novel − familiar | Exploration time in | the choice trial (s) = |
| Group | | (N − F)/(N + F) | object (s) | the sample trial (s) | N + F |
| G1-Control | Mean | 1.2 | 0.4 | 36.7 | 23.3 |
| N = 16 | SEM | 3.9 | 1.0 | 3.4 | 1.9 |
| p vs. Random≤ | | 0.767 | 0.703 | | |
| G2-Donep 2 | Mean | 19.3 | 4.3 | 20.2 | 21.7 |
| N = 16 | SEM | 3.2 | 0.9 | 3.4 | 2.5 |

| | | | | |
|---|---|---|---|---|
| p vs. Random≤ | 0.001 | 0.001 | | |
| p vs. G1-Control≤ | 0.002 | 0.007 | 0.002 | 0.616 |

### Effect of NLS-12

The results are presented in Table 3.

The discrimination index (DI, FIG. 2):
For the NLS-12 1 group: was not significantly different from 0, was not significantly different from that of the control group and tended to be lower than that of the Donep 2 group.
For the NLS-12 4 group: was significantly higher than 0 but was not significantly different from that of the control group and was not significantly different from that of the Donep 2 group.
For the NLS-12 8 group: was significantly higher than 0, was significantly higher than that of the control group and was not significantly different from that of the Donep 2 group.

The difference of exploration time between the novel object and the familiar object (N-F; FIG. 3):
For the NLS-12 1 group: was not significantly different from 0, was not significantly different from that of the control group and was not significantly different from that of the Donep 2 group.
For the NLS-12 4 group: was significantly higher than 0 but was not significantly different from that of the control group and was not significantly different from that of the Donep 2 group.
For the NLS-12 8 group: was significantly higher than 0, was significantly higher than that of the control group and was not significantly different from that of the Donep 2 group.
Summary. NLS-12 improved the recognition of the familiar object, i.e. improved memory. This effect was significant at 8 mg/kg and was not significantly different to that of donepezil (2 mg/kg). It might be present at 4 mg/kg and was not significant at 1 mg/kg.

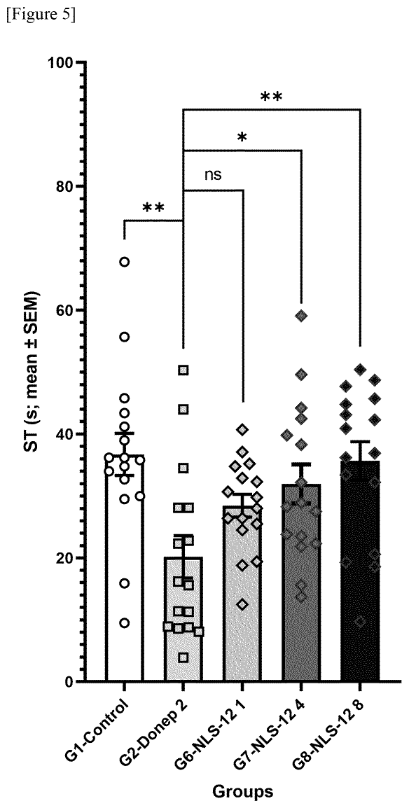

The exploration time during the sample trial (ST; 30 min post treatment):
For the NLS-12 1 group: was not significantly different from that of the control group (FIG. 4) and was not significantly different from that of the Donep 2 group (FIG. 5).
For the NLS-12 4 group: was not significantly different from that of the control group (FIG. 4) and was significantly higher than that of the Donep 2 group (FIG. 5).
For the NLS-12 8 group: was not significantly different from that of the control group (FIG. 4) and was significantly higher than that of the Donep 2 group (FIG. 5).

For NLS-12 1,4 and 8 groups, the exploration time during the choice trial (CT; 72 h post-treatment; FIG. 6): was not significantly different from that of the control group and was not significantly different from that of the Donep 2 group.
Summary. NLS-12 (1, 4, 8 mg/kg) did not significantly modify the exploration time 30 min post-treatment —contrary to donepezil (2 mg/kg)—and 3 days post treatment.

TABLE 3
Control and NLS-12 1, 4 and 8 groups. Indices of memory: discrimination index (DI) and difference
of exploration time between the novel object and the familiar object (N − F). Indices
of exploration: exploration time during the sample trial (ST) and during the choice trial
(CT). Results are expressed as mean and SEM. Statistical analyses: “p vs. random”,
difference vs. 0 (paired Student's t test; one-way ANOVAs for comparisons NLS-12 and Control
groups and for comparisons NLS-12 and Donep 2 groups; “p vs. G1-Control”, Dunnett's
test (except Donep 2 group: unpaired Student's t test); “p vs. G2-Donep 2”, Dunnett's test.

| | | DI | N − F | | CT |
|---|---|---|---|---|---|
| | | Discrimination | Exploration time | ST | Exploration time in |
| | | Index = 100 × | novel − familiar | Exploration time in | the choice trial (s) = |
| Group | | (N − F)/(N + F) | object (s) | the sample trial (s) | N + F |
| G1-Control | Mean | 1.2 | 0.4 | 36.7 | 23.3 |
| N = 16 | SEM | 3.9 | 1.0 | 3.4 | 1.9 |
| p vs. Random≤ | | 0.767 | 0.703 | | |
| G2-Donep 2 | Mean | 19.3 | 4.3 | 20.2 | 21.7 |
| N = 16 | SEM | 3.2 | 0.9 | 3.4 | 2.5 |
| p vs. Rondom≤ | | 0.001 | 0.001 | | |
| p vs. G1-Control≤ | | 0.002 | 0.007 | 0.002 | 0.616 |
| G6-NLS-12 1 | Mean | 2.5 | 0.6 | 28.4 | 24.5 |
| N = 16 | SEM | 5.9 | 1.5 | 1.8 | 2.1 |
| p vs. Random≤ | | 0.679 | 0.699 | | |
| p vs. G1-Control≤ | | 0.996 | 0.999 | 0.127 | 0.963 |
| p vs. G2-Donep 2≤ | | 0.058 | 0.127 | 0.130 | 0.746 |
| G7-NLS-12 4 | Mean | 12.9 | 3.7 | 31.9 | 25.6 |
| N = 16 | SEM | 5.7 | 1.4 | 3.2 | 3.0 |
| p vs. Random≤ | | 0.040 | 0.022 | | |
| p vs. G1-Control≤ | | 0.247 | 0.183 | 0.526 | 0.805 |
| p vs. G2-Donep 2≤ | | 0.691 | 0.974 | 0.018 | 0.524 |
| G8-NLS-12 8 | Mean | 22.5 | 5.9 | 35.6 | 25.8 |
| N = 16 | SEM | 4.3 | 1.2 | 3.1 | 1.9 |
| p vs. Random≤ | | 0.001 | 0.001 | | |
| p vs. G1-Control≤ | | 0.012 | 0.010 | 0.989 | 0.774 |
| p vs. G2-Donep 2≤ | | 0.946 | 0.699 | 0.002 | 0.492 |
| ANOVA | df | 3; 60 | 3; 60 | 3; 60 | 3; 60 |
| Control and NLS- | F | 3.918 | 4.252 | 1.644 | 0.264 |
| 12 groups | p | 0.013 | 0.009 | 0.189 | 0.852 |
| ANOVA | dF | 3; 58 | 3; 58 | 3; 60 | 3; 60 |
| Donep 2 and NLS- | F | 3.18 | 3.076 | 5.03 | 0.6152 |
| 12 groups | p | 0.031 | 0.035 | 0.004 | 0.608 |

### Additional Analyses

TABLE 4
Body weight (BW), exploration time of the novel object (N) and
exploration time of the familiar object (F). Statistical analyses:
“p vs. random”, difference vs. N vs. F (paired Student's
t test; “p vs. G1-Control”; other analyses; see Table 2-Table 4.

| | | | N | F |
|---|---|---|---|---|
| | | | Exploration | Exploration |
| | | BW | time of | time of |
| | | Body | novel | familiar |
| Group | | weight (g) | object (s) | object (s) |
| G1-Control | Mean | 22.5 | 11.8 | 11.5 |
| N = 16 | SEM | 0.4 | 1.0 | 1.1 |
| p vs. Random≤ | | | 0.703 | |
| G2-Donep 2 | Mean | 23.2 | 13.1 | 8.7 |
| N = 16 | SEM | 0.3 | 1.6 | 1.0 |
| p vs. Random≤ | | | 0.001 | |
| p vs. G1-Control≤ | | 0.510 | 0.529 | 0.058 |
| G6-NLS-12 1 | Mean | 22.8 | 12.6 | 12.0 |
| N = 16 | SEM | 0.4 | 1.3 | 1.2 |
| p vs. Rondom≤ | | | 0.699 | |
| p vs. G1-Control≤ | | 0.995 | 0.973 | 0.979 |
| p vs. G2-Donep 2≤ | | | 0.991 | 0.098 |
| G7-NLS-124 | Mean | 23.5 | 14.7 | 11.0 |
| N = 16 | SEM | 0.2 | 1.9 | 1.3 |
| p vs. Rondom≤ | | | 0.022 | |
| p vs. G1-Control≤ | | 0.126 | 0.379 | 0.982 |
| p vs. G2-Donep 2≤ | | | 0.820 | 0.322 |
| G8-NLS-12 8 | Mean | 22.9 | 15.9 | 9.9 |
| N = 16 | SEM | 0.3 | 1.3 | 0.9 |
| p vs. Rondom≤ | | | 0.001 | |
| p vs. G1-Control≤ | | 0.883 | 0.134 | 0.665 |
| p vs. G2-Donep 2≤ | | | 0.462 | 0.746 |
| ANOVA | df | | 3; 60 | 3; 60 |
| Control and NLS- | F | | 1.657 | 0.5794 |
| 12 groups | p | | 0.186 | 0.631 |
| ANOVA | dF | | 3; 60 | 3; 60 |
| Donep 2 and NLS- | F | | 0.9201 | 1.643 |
| 12 groups | p | | 0.437 | 0.189 |

### BIBLIOGRAPHY

1. Lennox G G, Lowe J S. Dementia with Lewy bodies. Baillieres Clin Neurol. 1997; 6(1):147-166.
2. Sadeghmousavi S, Eskian M, Rahmani F, Rezaei N. The effect of insomnia on development of Alzheimer's disease. J Neuro inflammation. 2020; 17(1):289. doi:10.1186/si2974-020-01960-9
3. Teaktong T, Piggott M A, Mckeith I G, Perry R H, Ballard C G, Perry E K. Muscarinic M2 and M4 receptors in anterior cingulate cortex: relation to neuropsychiatric symptoms in dementia with Lewy bodies. Behav Brain Res. 2005; 161(2):299-305. doi:10.1016/j.bbr.2005.02.019
4. Perry E, Court J, Goodchild R, et al. Clinical neurochemistry: developments in dementia research based on brain bank material. J Neural Transm (Vienna). 1998; 105(8-9):915-933. doi:10.1007/s007020050102
5. Piggott M A, Owens J, O'Brien J, et al. Muscarinic receptors in basal ganglia in dementia with Lewy bodies, Parkinson's disease and Alzheimer's disease. J Chem Neuroanat. 2003; 25(3):161-173. doi:10.1016/s0891-0618(03)00002-4
6. Roberson M R, Harrell L E. Cholinergic activity and amyloid precursor protein metabolism. Brain Res Brain Res Rev. 1997; 25(1):50-69. doi:10.1016/s0165-0173(97)00016-7
7. Roberson M R, Kolasa K, Parsons D S, Harrell L E. Cholinergic denervation and sympathetic ingrowth result in persistent changes in hippocampal muscarinic receptors. Neuroscience. 1997; 80(2):413-418. doi:10.1016/s0306-4522(97)00153-x
8. Rodriguez-Puertas R, Pascual J, Vilaró T, Pazos A. Autoradiographic distribution of M₁, M₂, M₃, and M₄ muscarinic receptor subtypes in Alzheimer's disease. Synapse. 1997; 26(4):341-350. doi:10.1002/(SICI)1098-2396(199708)26:4<341::AID-SYN2>3.0.CO;2-6
9. Teaktong T, Graham A J, Court J A, et al. Nicotinic acetylcholine receptor immunohistochemistry in Alzheimer's disease and dementia with Lewy bodies: differential neuronal and astroglial pathology. J Neurol Sci. 2004; 225(1-2):39-49. doi:10.1016/j.jns.2004.06.015
10. Ince P G, Perry E K, Morris C M. Dementia with Lewy bodies. A distinct non-Alzheimer dementia syndrome?Brain Pathol. 1998; 8(2):299-324. doi:10.1111/j.1750-3639.1998.tb00156.x
11. Bernard B A, Wilson R S, Gilley D W, Bennett D A, Fox J H, Waters W F. Memory failure in binswanger's disease and alzheimer's disease. Clin Neuropsychol. 1992; 6(2):230-240. doi:10.1080/13854049208401857
12. Reever C M, Ferrari-DiLeo G, Flynn D D. The M₅ (m5) receptor subtype: fact or fiction?Life Sci. 1997; 60(13-14):1105-1112. doi:10.1016/s0024-3205(97)00054-4
13. Garzón M, Pickel V M. Somatodendritic targeting of M₅ muscarinic receptor in the rat ventral tegmental area: implications for mesolimbic dopamine transmission. J Comp Neurol. 2013; 521(13):2927-2946. doi:10.1002/cne.23323
14. Steidl S, Miller A D, Blaha C D, Yeomans J S. M₅ muscarinic receptors mediate striatal dopamine activation by ventral tegmental morphine and pedunculopontine stimulation in mice. PLoS One. 2011; 6(11):e27538. doi:10.1371/journal.pone.0027538
15. Steidl S, Wasserman D I, Blaha C D, Yeomans J S. Opioid-induced rewards, locomotion, and dopamine activation: A proposed model for control by mesopontine and rostromedial tegmental neurons. Neurosci Biobehav Rev. 2017; 83:72-82. doi:10.1016/j.neubiorev.2017.09.022
16. Clader J W, Wang Y. Muscarinic receptor agonists and antagonists in the treatment of Alzheimer's disease. Curr Pharm Des. 2005; 11(26):3353-3361. doi:10.2174/138161205774370762
17. Lee J H, Koh S Q, Guadagna S, et al. Altered relaxin family receptors RXFP1 and RXFP3 in the neocortex of depressed Alzheimer's disease patients. Psychopharmacology (Berl). 2016; 233(4):591-598. doi:10.1007/s00213-015-4131-7

1-7. (canceled)

8. A method of prevention and/or treatment of neurodegenerative diseases when central muscarinic neurotransmission is compromised, comprising the administration of a compound of formula (I) 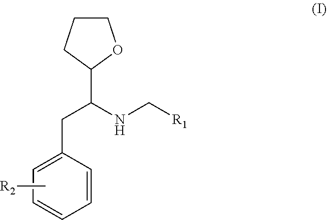
with R₁═H, —CH₃, or an acyl group, and
R₂═H or a halogen atom selected from the group consisting of F, Cl, Br, and I,
or a pharmaceutically acceptable isomer, salt, and/or solvate thereof, to a patient in need thereof, wherein the neurodegenerative disease is selected from the group consisting of Alzheimer's Disease, vascular dementia, Dementia with Lewy bodies, mixed dementia, frontotemporal lobar degeneration, and Parkinson's disease.

9. The method of claim 8, wherein R₁═—CH₃.

10. The method of claim 8, wherein R₂═H.

11. The method of claim 8, wherein R₁═—CH₃ and R₂═H.

12. The method of claim 8, wherein a therapeutic dose of 0.1 mg/kg/day to 400 mg/kg/day is administrated to the patient in need thereof.

13. The method of claim 12, wherein the therapeutic dose is 2 mg/kg/day to 128 mg/kg/day.

14. A method of prevention and/or treatment of neurodegenerative diseases when central muscarinic neurotransmission is compromised, comprising the administration of a pharmaceutical composition, the pharmaceutical composition comprising the compound of formula (I) as defined in claim 8 or a pharmaceutically acceptable isomer, salt, and/or solvate thereof, and a pharmaceutically acceptable excipient,
wherein the neurodegenerative disease is selected from the group consisting of Alzheimer's Disease, vascular dementia, Dementia with Lewy bodies, mixed dementia, frontotemporal lobar degeneration, and Parkinson's disease.

15. The method of claim 14, wherein the pharmaceutical composition comprises 1 mg to 80 mg of the compound of formula (I).

16. The method of claim 14, wherein the pharmaceutical composition comprises 2 mg to 40 mg of the compound of formula (I).

17. The method of claim 14, wherein the pharmaceutical composition is suitable for oral or parenteral administration.

18. The method of claim 14, wherein the pharmaceutical composition is in the form of a solution, a tablet, a capsule, or a Transdermal Delivery System.

19. The method of claim 18, wherein the pharmaceutical composition is in the form of an injectable solution.
